(19) United States
(12) Patent Application Publication
(10) Pub. No.: US 2021/0332315 A1
(43) Pub. Date: Oct. 28, 2021

(54) FILTRATION-CENTRIFUGE TUBE APPARATUS FOR HARVESTING ADIPOSE DERIVED STEM CELLS

(71) Applicants: Ben M. Khalaj, Lake Forest, CA (US); Siamak Agha-Mohammadi, Newport Coast, CA (US)

(72) Inventors: Ben M. Khalaj, Lake Forest, CA (US); Siamak Agha-Mohammadi, Newport Coast, CA (US)

(21) Appl. No.: 16/855,752

(22) Filed: Apr. 22, 2020

(51) Int. Cl. C12M 1/24; C12M 3/00; C12M 1/12; C12N 5/0775

(52) U.S. Cl. CPC C12M 23/08; C12M 23/46; C12M 23/06; C12N 5/0667; C12M 25/02

(57) ABSTRACT

The present disclosure relates generally to a tubing apparatus for separating and concentrating stem and stromal cells, also known as regenerative cells, from adipose tissue, more specifically to a defined process of extracting, separating and concentrating clinically useful regenerative cells from adipose tissue using a combination of mechanical disruption and filtration-centrifugation to obtain a highly enriched heterogeneous population of stem stromal cells. The centrifuge tube comprising a threaded top cap with male luer access port to be adapted to female luer of a syringe, a tapered main tubular barrel, a thin disk filter, and a bottom conical cap with luer access port for withdrawal of stem stromal cells via a syringe.

### FIELD OF THE INVENTION

The present invention relates to a device for separating and concentrating stem and stromal cells from adipose tissue. More particularly to obtain a highly enriched heterogeneous population of stem cells via a centrifuge tube for non-enzymatic processing.

### BACKGROUND OF THE INVENTION

Adipose tissue is an abundant source of mesenchymal stem cells, which have shown promise in the field of regenerative medicine. Furthermore, these cells can be readily harvested in large numbers with low donor-site morbidity. During the past decade, numerous studies have provided preclinical data on the safety and efficacy of adipose-derived stem stromal cells, supporting the use of these cells in future clinical applications. Various clinical trials have shown the regenerative capability of adipose-derived stem cells in subspecialties of medical fields such as plastic surgery, orthopedic surgery, oral and maxillofacial surgery, and cardiac surgery. In addition, a great deal of knowledge concerning the harvesting, characterization, and culture of adipose-derived stem cells has been reported.

In fat transfer procedures, after the fat cells are extracted from patient and collected in the syringe barrel, the fat cells goes through other important processes via a centrifuge tube such as process of separating and concentrating clinically useful regenerative cells from adipose tissue using a combination of mechanical disruption and filtration-centrifugation to obtain a highly enriched population of stem cells. The centrifuge tube has important role for these preparations.

A successful non-enzymatic method for harvesting adipose-derived stromal cells and adipose-derived stem cells from fat and lipo-aspirate is solely relays on centrifuge tubing and its filtration process in closed loop configuration. The centrifuge tube should be durable and reliable for centrifugation at 600 to 1800 rpm for about 5-10 minutes.

In this regards, the prior art centrifuge tube has wide opening at the upper end and also lack of access port at lower end. Furthermore, the tubing is not designed for lay down horizontal centrifugation forces as it is required for non-enzymatic method.

However, this invention overcomes the shortcomings of prior art and alleviates problems associated with tubing and substantially improve stem cell extraction and enrichments.

### SUMMARY OF THE INVENTION

The present disclosure relates generally to a tubing apparatus for separating and concentrating stem and stromal cells, also known as regenerative cells, from adipose tissue, more specifically to a defined process of extracting, separating and concentrating clinically useful regenerative cells from adipose tissue using a combination of mechanical disruption and filtration-centrifugation to obtain a highly enriched population of stem cells.

The centrifuge tube comprising a threaded top cap with male luer access port to be adapted to female luer of a syringe, a tapered main tubular barrel, a thin disk filter, and a bottom cap with lure access port for withdrawal of stem cells via a syringe.

It is the luer objective of this invention to minimize process time for non-enzymatic method of separating regenerative cells from adipose tissue and extracting the enriched stem stromal cells from conical cap access port for further cultivation and conditioning.

Further objects and advantages of this invention will become apparent from consideration of the drawings and descriptions that follow.

### BRIEF DESCRIPTION OF THE DRAWINGS

The drawings constitute a part of this specification and include exemplary embodiments to the invention, which may be portrayed in various forms. It is to be understood that in some instances various aspects of the invention may be exaggerated or enlarged to facilitate an understanding of the invention.

FIG. 1 is a side perspective view of an assembly of a filtration-centrifuge tube showing the present invention of preferred embodiment with 50 ml dose scale.

FIG. 2 is an exploded view of the tube as shown in FIG. 1.

FIG. 3 is a cross sectional view taken along line 3-3 in FIG. 2.

FIG. 4 is an assembled cross sectional view of shown in FIG. 1-3.

FIG. 5 is a fragmentary view taken from FIG. 4

FIG. 6 is similar view as FIG. 4 but showing that extracted lipoaspirate including fat is transferred into tube's barrel.

FIG. 7 is a plan view of horizontal centrifuge machine with nested six tubes for processing.

FIG. 8 is similar view as FIG. 6, but showing extracted stem stromal cells at the bottom of the tube after filtration and centrifugation of adipose.

FIG. 9 is similar view as FIG. 8 but showing the stem stromal cells are transferring into a syringe.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

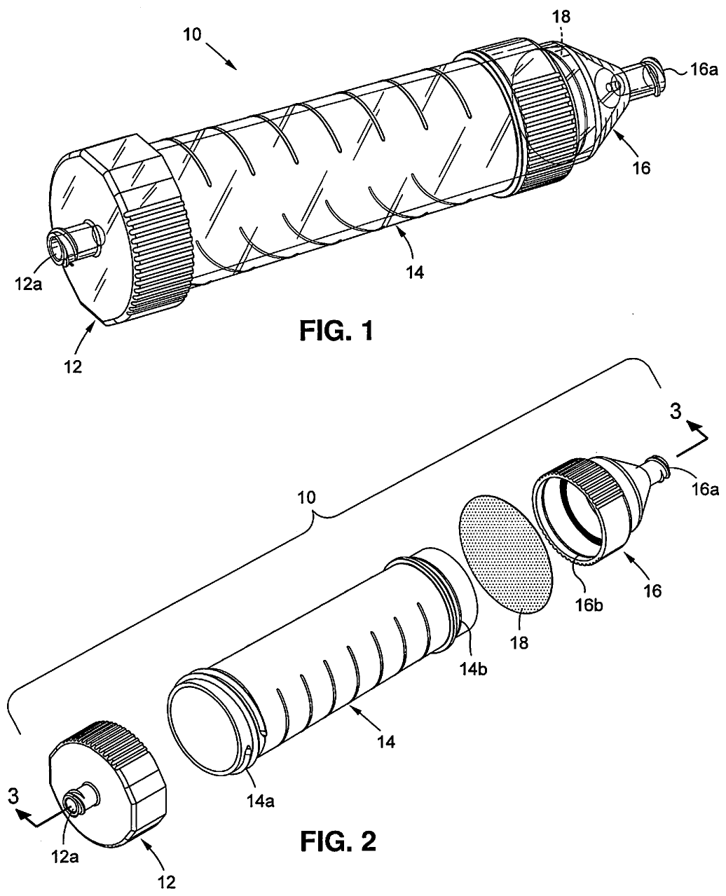

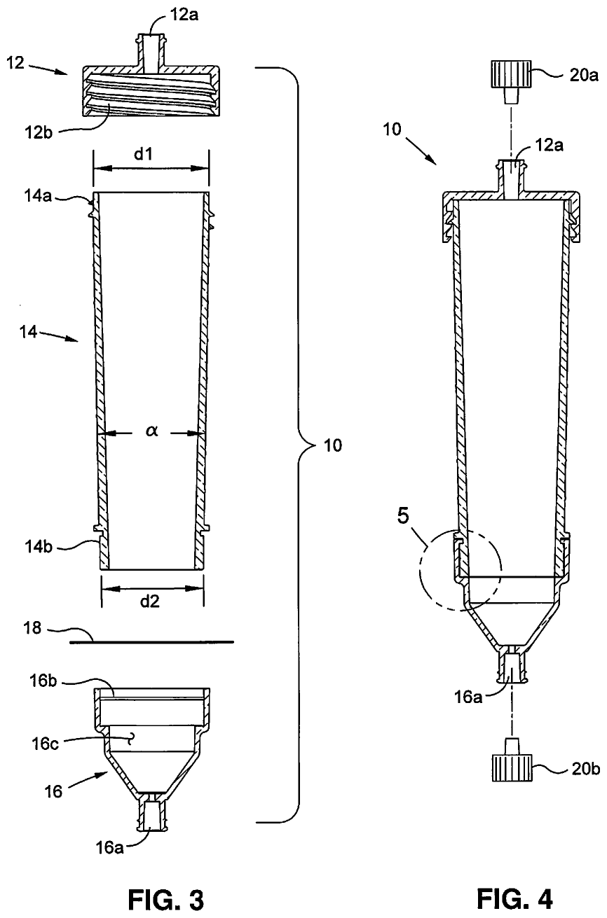

FIG. 1-4 illustrates a preferred embodiment of filtration-centrifuge tube 10 having 50 ml dose scale. However, the centrifuge tubes can range in size from 50 to 1000 ml. The tubing apparatus 10 comprises a threaded top cap 12 with male luer access port 12a to be adapted to female luer of a syringe 30, a tapered main tubular barrel 14 having external threads 14a and annular ring 14b, a thin disk filter 18 having at least 10 microns pore sizes, and a bottom conical cap 16 with luer access port 16a for withdrawal of stem cells via a syringe. The bottom cap is conical shaped and has a chamber 16c for collection of the stem cells. There are two threaded end plugs 20a, 20b to close the access ports 12a, 16a.

The thin disk filter 18 can be multi-filters or membranes or centrifuge tube strainers formed from any natural or synthetic polymers, paper, ceramics or metals such as stainless steel or nickel. Typically, nominal pore sizes of theses one or more filters or membranes range from about 10 microns to about 29 microns.

In some embodiments, the filters or membranes may be treated to contain antibodies/ligands which recognize select surface moieties that are specific to select cell types.

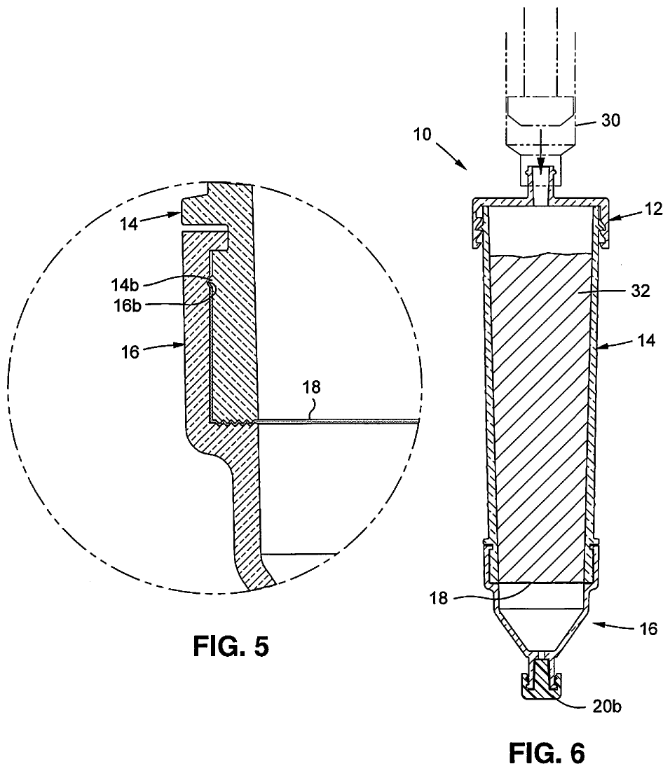

Now refereeing to FIG. 5, the disk filter 18 is placed in-between the main barrel 14 and the top of conical end cap 16. Furthermore, the filter 18 also tucked between the side walls. The inner portion of conical cap has a groove 16b that snaps tightly with annular outer ring 14b of main barrel 14. This tight connection between the conical cap 16 and main barrel 14 is to make sure the filter is secure and in placed so there would not be any slippage in high speed centrifugation processing.

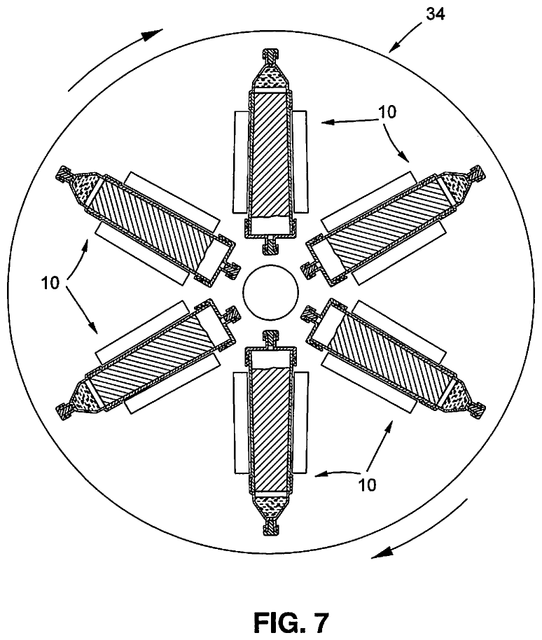

FIG. 6-9 are showing the usages of the tube in conjunction with non-enzymatic method of stem stromal cells extraction. In this method, the syringe 30 transfer lipoaspirate including adipose 32 into tube's barrel via its access port 12a as seen in FIG. 6. Then closing the access port 12a by using threaded plug 20a to prepare the tube 10 for horizontal centrifugation process 34.

The horizontal centrifugation should be at 600 to 1800 rpm for 5-10 minutes, adipose derived stem cells (ADSCs) passed through the filter 18. The physiological infiltration fluid (PIF) was then removed from the stem stromal cells within the filter. In this fashion, the infiltration fluid containing fluid, red blood cells, oil, and broken cell debris was separated from the centrifuged ADSCs. By reducing the g-force to about 100 to 600 rpm for 5 to 10 minutes, while the ADSCs are centrifuged through the filter, the majority of the red blood cells and debris remain within the strainer. This is of importance, since the ADSC viability is inversely related to the number of the red blood cells within the final fraction of ADSCs that is to be used for clinical applications.

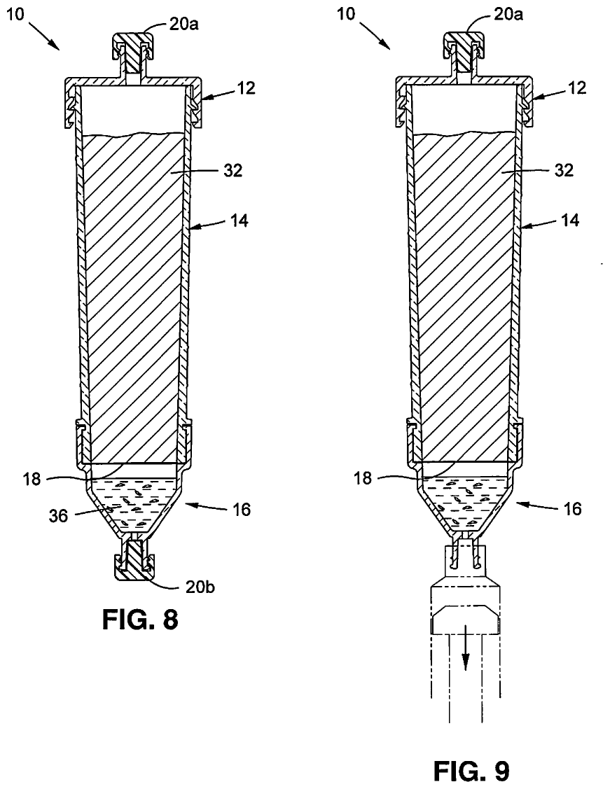

FIG. 8-9 are showing the collected and redefined stem stromal cells 36 is stored in conical chamber 16c at the bottom of conical cap 16. Then stem stromal cells 36 can be withdrawn via access port 16a using a syringe to be used in clinical applications.

While this invention is susceptible to embodiments in many different forms, this specification and the accompanying drawings disclose only some specific forms as examples of the invention. The invention is not intended to be limited to the embodiments as described; however, the scope of the invention is pointed out in the appended claims.

We claim:

1- A tubing apparatus for obtaining highly enriched population of stem cells via centrifuge comprises;
1- A tapered tubular barrel, the barrel main body having a top opening with threaded end and bottom opening end, the main body tapered toward the bottom end;
a thin disk filtration member;
a top cap with lure access port, the top cap is threaded and placed over the top opening end of the main body; and,
a bottom conical cap with lure access port, the bottom cap is securely press fitted at the bottom end of the main tubular barrel with the thin disk filtration member sandwiched in between.

2- A tubing apparatus for obtaining highly enriched population of stem cells via centrifuge according to claim 1, wherein said a thin disk filtration member has nominal pore sizes from about 10 microns to about 250 microns.

3- A tubing apparatus for obtaining highly enriched population of stem cells via centrifuge according to claim 1, wherein said a thin disk filtration member is made of synthetic polymers.

4- A tubing apparatus for obtaining highly enriched population of stem cells via centrifuge according to claim 1, wherein said a thin disk filtration member is made of synthetic paper.

5- A tubing apparatus for obtaining highly enriched population of stem cells via centrifuge according to claim 1, wherein said a thin disk filtration member is made of ceramic.

6- A tubing apparatus for obtaining highly enriched population of stem cells via centrifuge according to claim 1, wherein said a thin disk filtration member is made of stainless steel.

7- A tubing apparatus for obtaining highly enriched population of stem cells via centrifuge according to claim 1, wherein said a thin disk filtration member may combine plurality of disk filtration members.
